(19) United States
(12) Patent Application Publication
(10) Pub. No.: US 2017/0105675 A1
(43) Pub. Date: Apr. 20, 2017

(54) Cardiac function monitor and/or intervention system attached outside or inside of heart

(71) Applicant: Xiaohui Zhou, Nanjing (CN)

(72) Inventors: Xiaohui Zhou, Nanjing (CN); Lei Han, Nanjing (CN); Yuyan Zhou, Nanjing (CN); Li Zhou, Nanjing (CN); Guohua Li, Nanjing (CN); Yongzhen Li, Nanjing (CN)

(21) Appl. No.: 15/394,852

(22) Filed: Dec. 30, 2016

(30) Foreign Application Priority Data
Aug. 31, 2016 (CN) 201610781862.1

(51) Int. Cl. A61B 5/00; A61B 5/0402; A61B 5/0205; A61B 5/01; A61N 1/362; A61B 5/02; A61N 2/00; A61H 9/00; A61M 5/31; A61M 39/10; A61N 2/02; A61B 5/145

(52) U.S. Cl. CPC A61B 5/4836; A61N 2/02; A61B 5/0402; A61B 5/02055; A61B 5/01; A61B 5/14539; A61B 5/14503; A61B 5/02028; A61N 2/004; A61H 9/00; A61M 5/31; A61M 39/10; A61N 1/362; A61B 5/026

(57) ABSTRACT

A surface attached cardiac function monitor and/or intervention system comprises of a cardiac support device, a cardiac function monitor device and/or intervention device. Cardiac support device is attached on an external or internal surface of a cardiac chamber and supports it. The cardiac function monitor device is connected with a biochemical and physiological sensor. The physiological and biochemical sensor transmits variations of biochemical and physiological parameters that are received by the cardiac function monitor device. The intervention device has at least one member selected from pressure intervention device, an electrical/magnetic stimulation intervention device and a medicine intervention device. This medical system of the present invention could help in diagnosis as well as treatment of the heart failure and other myocardial diseases to improve the condition of patient. It could also be helpful for the monitoring, diagnosis and treatment of diseases of lungs, kidney, liver, spleen, stomach and bladder, etc.

### CROSS REFERENCE OF RELATED APPLICATION

The present application claims priority under 35 U.S.C. 119(a-d) to CN 201610781862.1, filed Aug. 31, 2016.

### BACKGROUND OF THE INVENTION

### Area of Invention

The present invention relates to the technical field of medical devices, and more particularly to a medical device used for the monitoring, diagnosis and treatment of heart failure and other myocardial diseases. It could also be helpful for the diagnosis and treatment of diseases of lungs, kidney, liver, spleen, stomach and bladder.

### DESCRIPTION OF RELATED ARTS

Heart failure, a clinical syndrome of ventricular filling or ejection abnormality caused by disorders in cardiac structure and function, is a common pathological state of the heart. The basic manifestations of heart failure are dyspnea, fatigue, limited exercise tolerance and fluid retention, wherein the fluid retention may further lead to pulmonary congestion and peripheral edema.

Although progress has been made in recent years in basic and clinical research on prevention, diagnosis and treatment of heart failure, 50% of the patients died from heart failure in 3 years.

The prevention and diagnosis of heart failure is mainly based on the monitoring of cardiac function and physiological parameter parameters. Currently, most of the cardiac function tests are based on monitoring of extra-corporal non-invasion cardiac physiological parameters, like body surface electrocardiograph (ECG), ultrasonic cardiogram, CT, nuclear magnetic resonance etc. These techniques are advantageous as they are noninvasive and could be easily monitored dynamically and continuously. However, these techniques indirectly and integrally reflect cardiac function and physiological condition, but are poor in intuitive, microcosmic and accurate reflection. Internal invasive monitoring method includes intracavitary ECG, three-dimensional (3D) cardiac electro-mechanical detecting system (NOGA) and cardiac catheter intervention monitor and has advantages of intuition and relative precision, but an invasive operation is required at every time of monitoring. In addition, the invasive operation must be ended with the termination of the monitoring and a long-term and dynamic monitoring cannot be achieved. This limits the clinical significance of these devices.

Heart transplantation is an effective way to treat end-stage heart failure. However, it is not feasible due to short supply of donors and limitation of social, economic and technical factors, therefore different techniques developed to treat end stage heart failure. Mechanotherapy is the recent technique and mainly includes: left ventricular-assist device (LVAD), a cardiac support device (CSD), a quantitative Ventricular Restraint technique (QVR) and Heartnet Ti—Ni alloy heart tube-network, etc.

LVAD is a mechanical pump that regulates ventriculus sinister function and increases cardiac output. In 1960s, LVAD was used as a transitional treatment before cardiac transplantation to a plurality of European and American peoples as an alternate treatment of end-stage heart failure. LVAD mainly regulates the left ventricular systolic dysfunction, but it can't restore shape and function of the heart. This is an expensive procedure as surgery is required to implant this device. Currently, the research on LVAD has been extended from primarily aspects of physiology and biochemistry, morphology and neuroendocrine to a deep molecular level of myocardial cell matrix metabolism and myocardial protein expression. A series of clinical researches conducted on LVAD provided satisfactory results. Thus, the US Food and Drug Administration approved it for the treatment of heart failure. However, there are some problems associated with LVAD due to its not being used extensively. Surgical cost of LVAD implantation and a continuous medical cost for a postoperative protection are high. LVAD requires a continuous external power, serving as a driving force in order to work normally, and the strong external electric and magnetic fields may cause damage to the electrical & mechanical malfunction of LVAD. Moreover, external power supply or other circulating auxiliary equipment limits the activity of the patient to reduce his quality of life. Other potential risk factors include gas embolism, infection, thromboembolism, hemolysis, etc.

Cardiac support device (CSD), a device used to treat heart failure is a tube-network adhered firmly and uniformly on a surface of epicardium. Long-term implantation of CSD tends to return the ventriculus sinister to a normal state, however the current clinical trials indicate no definite therapeutic effects of CSD until now.

Recently, new devices like Heartnet, QVR etc have been developed for treatment of heart failure. Heartnet is a highly elastic nickel-titanium alloy network that binds directly on the external surface of the heart and improves the ventricular systolic function. QVR is a semi-ellipsoidal balloon made of medical grade polyurethane, and regulates the ventricular pressure by controlling the flow of gas into the balloon. Study shows that these are effective methods for heart failure treatment, but they are still in a preclinical experimental research stage.

Fore mentioned devices mainly focus on regulating ventricular pressure and restricting cardiac enlargement. Advancement in basic clinical application and basic research revealed that these devices have certain limitations in application as below.

1. Passive Treatment and Regulation

### CSD and Heartnet are not Capable of Performing an Initiative Clinical Intervention.

They are difficult to be regulated and controlled once they have been implanted in the body. These equipments rely on the structure and natural properties of their materials, and a dynamic, quantitative, time-set, real-time, opportune and timely regulation is not possible.

2. Monotony in Therapeutic Effect

The LVAD, CSD, Heartnet and QVR can't be used in conjunction with other medical methods. In particular, it is difficult to use the LVAD, CSD, Heartnet and QVR directly and effectively with other medicine intervention, treatment or electromagnetic stimulation.

3. Limitations of Therapeutic Extension

Around the world, new and on-going researches have provided potential treatment solutions for heart failure such as stem cell repair treatment, gene repair, immunobiology treatment, cardiac radiofrequency ablation, low temperature plasma ablation surgery etc. Heart failure treatment devices will have a broader application prospects if they are used in conjunction with these potential treatments. However, The LVAD, CSD, Heartnet and QVR can't be used effectively with fore-mentioned treatments in a convenient, fast and economic way. It limits the use of LVAD, CSD, Heartnet and QVR.

So it is of great clinical importance if a new device invented for heart failure treatment is free from shortcomings such as passive regulation, monotony limitation in therapeutic effect and therapeutic expansion.

### SUMMARY OF THE PRESENT INVENTION

Keeping in view of the disadvantages of the conventional devices, the present invention provides an attached cardiac function monitor and/or intervention system, for direct and precise monitoring of physiological and biochemical parameters of local endocardium/epicardium, directly and precisely positioning the local endocardium/epicardium for drug administration, ventricular pressure regulation, and electrical/magnetic stimulation. Current invention works on principle of combining monitoring and treatment, and incisively monitors the state of the cardiac function to improve the condition of patients by multiple treatment methods.

The technical solution of the new invention is as follows.

A surface attached cardiac function monitor and/or intervention system comprises: a cardiac support device and a cardiac function monitor device and/or an intervention device;

wherein the cardiac support device adheres on internal or external surface of an atrium or a ventricle and supports them;
the cardiac function monitor device is connected with a physiological and biochemical sensor;
at least one component of the new invention is selected from a pressure intervention device, an electrical/magnetic stimulation intervention device or a medicine intervention device; wherein the pressure intervention device comprises a liquid delivery tube and a liquid perfusion device; the electrical/magnetic stimulation intervention device comprises an intervention or stimulation electrical/magnetic electrode and a power output device; the medicine intervention device comprises a microsyringe;
one component selected from the physiological and biochemical sensor, the liquid delivery tube, the intervention or stimulation electrical/magnetic electrode and the microsyringe is embedded in the tube walls, filled in the aperture of the tube walls or adhered on an internal or external surface of the cardiac support device.

The physiological and biochemical sensor of this invention transmit signals via wire (or wireless) to the cardiac function monitor device.

The power output device independently and selectively control one or more stimulating electrical/magnetic electrode.

The microsyringe is connected with a separate medicine delivery tube, and selectively used in diseased region.

The liquid delivery tube execute area distribution according to a clinical treatment solution, wherein the liquid delivery tube in each area is relatively independent, therefore pressure is selectively applied on different areas.

Preferably, the cardiac support device of the present invention is a cardiac tube-network.

Cardiac support device is a solid and end-sealed tube network. The fore mentioned medicine delivery tube, wires of microsyringe, the physiological and biochemical sensor or the intervention or stimulation electrical/magnetic electrode spreads along the tube network on an internal or an external side of the cardiac tube-network, and then extends out of the human body via a subcutaneous tunnel.

Cardiac tube-network is an end-sealed tube network made of hollow tubes, which are completely communicated or form a plurality of independent areas, and the interior of each independent area is intercommunicating while the independent areas are not communicating with each other and the cardiac tube-network has at least one open end extending out of human body. These hollow tubes could serve as a liquid delivery tube for a pressure intervention device and the cardiac tube-network is connected with a liquid perfusion device out of human body via tubes at end. These tubes could also serve as wires of physiological and biochemical sensor, the intervention or stimulation electrical/magnetic electrode or as channels for the medicine delivery tube of the microsyringe. The wires or the medicine delivery tube then could be connected to a device out of human body through the end of cardiac tube-network via subcutaneous channel.

The cardiac tube-network of this new device is preferred a cardiac tube-network revealed in a Chinese patent application with an application number of CN200910031330.6.

The physiological and biochemical sensor transmits signal variation of physiological parameters detected on an internal or an external surface of a heart to the cardiac function monitor device in vitro; these physiological and biochemical parameters include cardiac-electric induction, PH value, temperature, color, cardiac wall tension, cardiac chamber internal pressure, flow and hemodynamic parameters.

The physiological and biochemical sensor, the intervention or stimulation electrical/magnetic electrode or the microsyringe of the present invention could be distributed by 1˜10³⁰/cm², wherein a distribution location might be inside the tube channel of the cardiac tube-network or inside/outside of the tube wall close to myocardial cells. The physiological and biochemical sensor, the intervention or stimulation electrical/magnetic electrode and the microsyringe are distributed proportionally as 1:1:1 or other proportions.

The cardiac function monitor device of present invention is selected from a commonly used clinical ECG monitor or multi-channel physiology recorder, such as Mindray monitor, Bollen ECG monitor, SI CHUAN monitor, Siemens monitor, Keliwei monitor, World emperor ECG monitor, Futian ECG monitor, Li Bang ECG monitor, Rui Bo ECG monitor, Neusoft ECG monitor.

Physiological and biochemical sensor may be a tension sensor, pressure sensor, PH sensor, color sensor, temperature sensor, flow sensor or a cardiac-electric conduction electrode. Size of the physiological and biochemical sensor is preferably within a range of 1 nm˜100 μm. Sensitivity of the tension sensor is preferably within a range of 10⁻¹⁰˜10¹⁰ Newton. Sensitivity of the pressure sensor is preferably within a range of 10⁻¹⁰˜10¹⁰ pa. Sensitivity of the PH sensor is preferably within a range of 10¹⁰˜10¹⁰. A sensitivity of the color sensor and the temperature sensor is preferably within a range of 10⁻¹⁰˜10¹⁰ nm of optical wave. A voltage sensitivity of the cardiac-electric conduction electrode is preferably within a range of 10⁻¹⁰˜10¹⁰ V. A magnetic induction sensitivity is preferably within a range of 10⁻¹⁰˜10¹⁰ Tesla. A sensitivity of the flow sensor is preferably within a range of 10⁻¹⁰˜10¹⁰ L/min.

An output of the electrical/magnetic stimulation intervention device of the present invention is electric or electromagnetic energy.

The liquid delivery tube and the liquid perfusion device initiatively and controllably apply hydraulic pressure to the heart. Liquid used may be:

Normal saline;
Conventional polarized liquid with a formulation of: 500 ml of 10% glucose+10 U of insulin+10 ml of 10% potassium chloride;
Magnesium polarized liquid with a formulation of: 500 ml of 10% glucose+10 U of insulin+10 ml of 10% potassium chloride+10-20 ml of 10% magnesium sulphate;
Enhanced polarized liquid with a formulation of: 500 ml of 10% glucose+10 U of insulin+10 ml of 10% potassium chloride+20 ml of L-aspartic acid potassium magnesium (L-PMA);
High concentration polarized liquid with a formulation of: 20 U of insulin+15 ml of 10% potassium chloride+500 ml of 10% glucose solution and 60 ml of 50% glucose;
Simplified polarized liquid with a formulation of: 20 ml of L-aspartic acid magnesium potassium and 500 ml of 10% glucose solution;
Energy mixture with a formulation of: 500 ml of 10% GS+40 mg of ATP+100 u of coenzyme A +0.4 inosine;
Lyticcocktail with a formulation of: pethidine 100 mg+chlorpromazine 50 mg+promethazine 50 mg;
Dehydration mixture with a formulation of: 125-250 ml of 20% mannitol+5-10 mg of dexamethasone;
Fructose sodium diphosphate injection; and 5% glucose injection;

When the cardiac support device is adhered on an external wall of the atrium/ventricle, the hardness of an external side wall of the liquid delivery tube is preferably 1.5 times or more than the hardness of the internal side wall. Similarly if it is adhered on an internal surface of the cardiac chamber, the hardness of an internal side wall the liquid delivery tube is preferably 1.5 times or more than the hardness of the external side wall.

Both the perfusion device and the medicine loading device in new invention deliver liquid or medicine by constant pressure or a pump.

An output voltage of the intervention or stimulation electrical electrode is preferably 10⁻¹⁰˜10¹⁰V; output magnetic field intensity of the intervention or stimulation magnetic pole is 10⁻¹⁰˜10¹⁰ Tesla. A pinhole diameter of the microsyringe is preferably 10⁻¹⁰˜10⁷ nm.

The intervention or stimulation electrical/magnetic devices initiatively, quantitatively and controllably emit current or magnetic field radiation pulse for intervening cardiac electrophysiological functions. The microsyringe releases substances for intervening cardiac electrophysiological functions. These substances could be monomeric compound, plant extract, traditional Chinese medicine injection, polypeptide fragments, small molecular protein, macromolecule protein or bone marrow/embryonic stem cells etc.

According to a preferred embodiment, a surface attached cardiac function monitor system comprises a cardiac tube-network and a cardiac function monitor device connected with a physiological and biochemical sensor, so as to achieve a real-time monitor of the cardiac function.

According to another preferred embodiment of the present invention, a surface attached cardiac function monitor system comprises a cardiac tube-network and a cardiac function monitor device, and provides functional intervention in time when the heart functions are abnormal.

According to another preferred embodiment of the present invention, a surface attached cardiac function monitor system comprises a cardiac tube-network and a cardiac function monitor device connected with a physiological and biochemical sensor, which not only provide real-time monitor of the cardiac function before treatment and functional intervention of heart in time, but also monitors the cardiac function of the patient after treatment. It results in real-time feedback information for identifying treatment effect or regulating treatment solution.

The medicine used in device is selected from the groups like: diuretic, cardiotonic, angiotensin-converting enzyme inhibitor, angiotensin II receptor blocker, β-adrenergic blocker, anticoagulant, vasodilator, anti-myocardia ischemia drug, coronary-dilating drug drug and stem cells. At least one kind of the medicine is preferably selected from the group consisting of:

sodium ferulate injection, esmolol hydrochloride injection, composite salvia miltiorrhiza injection, ligustrazine injection, breviscapine injection, safflower injection, shuxuening injection, buflomedil hydrochloride injection, puerarin injection, ginkgo dipyridamole injection, ligustrazine glucose injection, astragalus injection, Shenmai injection, nitroglycerin injection, isosorbide dinitrate injection, low molecular weight heparin calcium injection, fibrinolysis enzyme for injection, defibrase for injection, urokinase for injection, cardiac stem cells, bone marrow stem cells and embryonic stem cells. A preferred stem cell treatment regimen is 10⁵-10²⁰ bone marrow stem cells per day, which is administered continuously for 1 to 60 days.

According to a preferred embodiment, the cardiac support device is made of conductive hydrogel, silica gel or degradable biocompatible materials. The hydrogel material is conductive and could partially or completely replace effects of cardiac-electric conduction electrode, so as to transmit electric signals on a surface of the heart via wires to a receiver or device outside the body. Meanwhile, functions of pressure sensor, PH sensor, color sensor, temperature sensor and flow sensor cannot be replaced, and must be accomplished by corresponding sensors.

Cardiac tube-network could be prepared with the help of computer software and hardware considering specific conditions such as sizes of heart of individual patient. The cardiac tube-network is preferably directly printed utilizing materials such as silicone and conductive hydrogel by a three-dimensional printing technique. Alternatively, cardiac tube-network could also be prepared by a method including preparation of a solid tube structure by a three-dimensional printing technique, followed by covering with a flexible material such as silica gel, and finally remove the coated solid tube structure by physical or chemical means without damaging to the structure.

The cardiac tube-network is manufactured by a process comprising following steps:

\textcircled{1} preparing a solid structure of the device by 3D printing device using different type of waxes like blue, green, red, black and white wax;
\textcircled{2} soaking the solid structure of the blue wax into liquid silicone, latex, conductive hydrogel, silicone adhesive, rubber or polymer plastic material for is to 24 hours;
\textcircled{3} coating with curing agent to form a membrane shaped structure; or expose it to a temperature ranges from 0-10000° C. for is to 240 hours to cure;
\textcircled{4} removing the solidified waxy solid material in the device after curing in such a manner that the membrane shaped structure is turned to hollow and interconnected tubular network structure;
\textcircled{5} washing membrane shaped structure with a solvent, in such a way that an inner and outer surface of membrane becomes more smooth and soft; and
\textcircled{6} doing additional surface treatment to improve smoothness of the inner and outer surface, flexibility and mechanical strength.

This device is not only used for monitoring and treating heart disease; but it could also be used on an external surface of lungs, kidney, liver, spleen, stomach, bladder, brain or spinal cord for diagnosis and treatment of emphysema, renal and hepatic failure, functional or structural abnormalities of the spleen and stomach, diseases of urinary retention, central nervous system dysfunction such as multiple sclerosis, cerebral hemorrhage, cerebral infarction and epilepsy respectively.

When the surface attached cardiac function monitor or intervention system of the present invention is being utilized, it is enclosed or adhered on an external surface of the heart, or affixed on endocardium by surgery and then the incision of the surgery would be sewed up. Wires of the physiological and biochemical sensor, the intervention or stimulation electrical/magnetic electrode, liquid delivery tube or tubes of the microsyringe are connected to epidermis of human body via a subcutaneous tunnel, and a constant or permanent joint is kept in the epidermis which helps temporarily or permanently or constantly in connection with such devices outside the body as cardiac-electric monitor device, multi-channel physiology recorder, electrical/magnetic power output device.

Beneficial effects of the present invention are as follows:

(1) Onset time of cardiac disease to be timely detected and predicted is an important factor in treating cardiac diseases. Conventional monitoring device can't achieve a long-term and round-the-clock monitoring of heart function so it is difficult to predict and diagnose the occurrence and development of cardiac disease in time. This new device of the present invention is capable of performing a long-term, precise, real-time, dynamic and round-the-clock monitoring of cardiac function, and thus is superior to the conventional cardiac physiological and biochemical monitoring method.

As soon as cardiac support device is implanted on an external or internal surface of the cardiac chamber, the process of the implanting could be terminated, thus long-term, continuous, dynamic, real-time, accurate and in situ cardiac function monitor is started and achieved under a minimally invasive condition. In addition, the cardiac physiological parameters detected by the monitor system of present invention could serve as a negative feedback signal that could precisely control the resultant effects of treatment such as therapeutic substance release, electrical stimulation intensity, or magnetic field stimulation intensity.

(2) Conventionally, cardiac disease are treated with surgical treatments such as heart transplantation or coronary artery bypass surgery, cytological treatment including cardiac stem cell transplantation, in vitro or in vivo defibrillation, or medicine administration on regional myocardial or the whole body. Limitations of these methods include a low treatment precision, and they are only capable of intervening the function and monitoring of physiological and biochemical parameters in an organ or tissue level, while intervening and monitoring at cellular level is not possible. This new invention helps in monitoring or precisely adjusting the physiological and biochemical parameters targeting on one or more abnormal myocardial cells. Moreover, it doesn't only monitor physiological and biochemical parameters on single cell, but also helps in local administration and cell transplantation in or out of the cardiac chamber, delivering electrical/magnetic stimulation. It is thus a multi-functional device to treat heart diseases more effectively than conventional devices, which change the present medical treatment scheme for cardiac diseases.

Presently, monitoring and treatment of the cardiac function are two separate sections. But this device monitors the cardiac physiological and biochemical parameters, moreover execute electrophysiological intervention or stimulation and the microamounted and precise positioning injection system simultaneously. It not only warns heart failure timely, but also delivers medicine instantly. In addition, it monitors heart function after the treatment in real time and send feedback signals to electrophysiological intervention or stimulation or a microamounted and precisely positioning injection system to regulate treatment intensity or solution. Moreover it monitors the condition of heart and identifies the treatment effects, thus helps the doctor to regulate and modify the clinical treatment solutions.

This innovative device of the present invention could also be used for the diagnosis and treatment of disease in other organs such as lungs, kidney, liver, spleen stomach and bladder etc.

The following detailed description, drawings and the appended claims gives us detailed understanding of objectives, features, and advantages of the device of the present invention.

### BRIEF DESCRIPTION OF THE DRAWINGS

FIG. 1 is a sketch view of a surface attached cardiac function monitor system according to a preferred embodiment of the present invention.

FIG. 2 is a section view of a tube of a cardiac tube-network attached with a physiological and biochemical sensor.

FIG. 3 is a sketch view of the cardiac function monitor system attached with multiple types of sensors.

FIG. 4 is a sketch view of a surface attached hydraulic-type cardiac function intervention system.

FIG. 5 is a sketch view of a surface attached electrical/magnetic-stimulation cardiac function intervention system.

FIG. 6 is a sketch view of a surface attached drug-delivery-type cardiac function intervention system.

FIG. 7 is a sketch view of a surface attached cardiac function monitor and intervention system.

### DETAILED DESCRIPTION OF THE PREFERRED EMBODIMENT

A concrete process of the present invention is illustrated combining with the preferred embodiments. It is understood that the embodiment of the present invention as is shown in the drawings and described in the words is exemplary rather than limiting.

Objectives of this new invention have been fully and effectively explained. Its embodiments have been shown and described for the purposes of illustrating the functional and structural principles and is subject to change without departure from such principles. Therefore, this invention includes all modifications encompassed within the spirit and scope of the following claims.

In the preferred embodiments below, the conventional methods and processes are not described in detail.

Further description of the present invention is illustrated by combining with the preferred embodiments.

Materials, reagents, apparatuses, equipment and etc. mentioned in the preferred embodiments below are all commercially available if no specific description is made.

### Embodiment 1

### Preparation of a Cardiac Tube-Network

It comprises following steps:

(1) performing computer-aided design (CAD) modeling using a conventional method in the field, wherein designs could be derived from digitized image reconstruction on a heart of a patient, e.g., image data could be obtained from finely layered three-dimensional reconstruction or scanning techniques like MRI or CT;
(2) by utilizing liquid silicone, latex, conductive hydrogel, silicone, rubber or polymer plastic materials, printing the cardiac tube-network by a three-dimensional printing technology;
Or alternatively,
\textcircled{1} manufacturing a solid structure of the device by a 3D printing device utilizing different materials like blue, green, red, black and white wax;
\textcircled{2} soaking the solid structure of the blue wax into liquid silicone, latex, conductive hydrogel, silicone, rubber or polymer plastic material for 1 s to 24 hours;
\textcircled{3} coating with curing agent to form a membrane shaped structure; or it is exposed to a temperature ranging from 0 to 10000° C. for is to 240 hours to be cured;
\textcircled{4} after curing, removing the solid material in the device such as solidified blue wax, in such a way that the membrane shaped structure turns to a hollow and interconnected tubular network structure.
\textcircled{5} washing membrane shaped structure with a solvent, in such a way that an inner and outer surface of membrane becomes smooth and soft.
\textcircled{6} doing additional surface treatment to improve smoothness of the inner and outer surface, and the flexibility and mechanical strength of the whole structure of the device of the present invention.

### Embodiment 2

A surface attached cardiac function monitor and/or intervention system comprises of a cardiac support device and a cardiac function monitor device and/or a cardiac function intervention device;

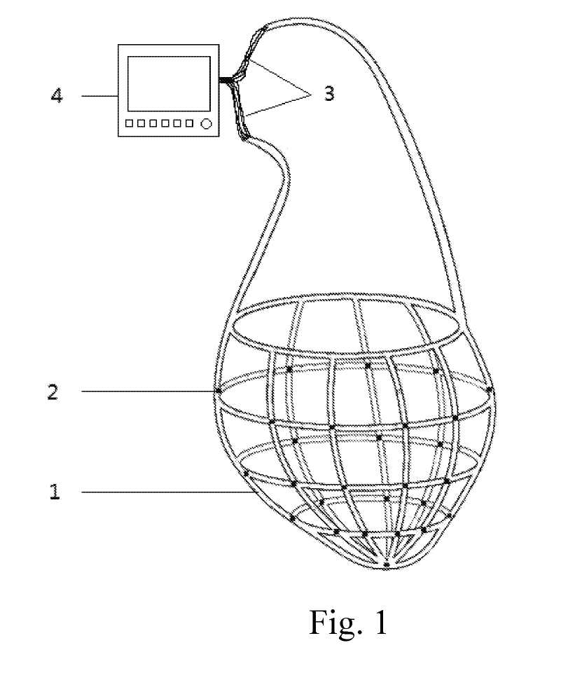

The cardiac support device is a cardiac tube-network. Structure is shown in FIG. 1, 1—cardiac tube-network; 2—physiological and biochemical sensor, 3—wire, 4—cardiac function monitor device.

The cardiac support device is attached on an external surface of a ventricle or an atrium or adhered on an internal surface of the cardiac chamber. The cardiac function monitor device is connected with the physiological and biochemical sensor which could be embedded in the tube walls, filled in the aperture of the tube walls or adhered on an internal or external surface of the cardiac support device.

### Embodiment 3

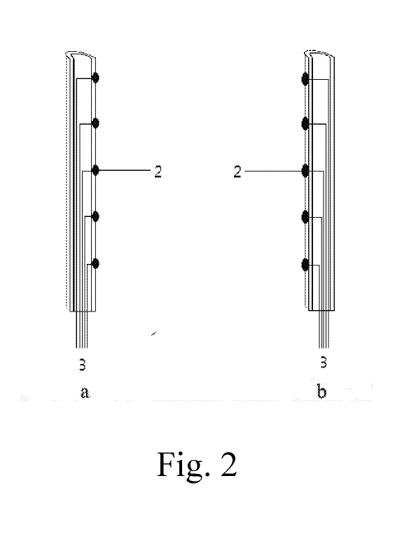

The basic structure is identical to the embodiment 2. Tube-network is composed of hollow tubes. All the hollow tubes are completely communicated or form a plurality of independent regions. It is intercommunicated within the region, and is not communicated between the regions. The wire of the physiological and biochemical sensor passes through the hollow tube of the tube-network and connects the function monitor device on one end of the tube-network. The structure is shown in FIG. 2, when the tube-network is attached on an external surface of the ventricle/atrium, the physiological and biochemical sensor is adhered on an internal side (see FIG. 2a) of the tube-network; and when the mesh is adhered on an internal surface of the internal cardiac chamber, the physiological and biochemical sensor is adhered on an external side of the tube-network (see FIG. 2b).

### Embodiment 4

The basic structure is identical to the embodiment 2 and embodiment 3. pressure sensors with various sizes within range of 1 nm-100 μm are adhered on an internal or an external side of the tube-network. The sensitivity of the pressure sensor ranges from 10⁻¹⁰ to 10¹⁰ pa. The sensor senses levels or intensity of the pressure on a surface of the cardiac chamber, and transmits signals via a wire in a hollow tube inside the tube-network to a multi-channel recorder, to achieve a real-time, dynamic and continuous monitoring of the surface pressure or intensity of the pressure in cardiac chamber, furthermore, indirectly deduce the level and variation of the internal pressure in the cardiac chambers.

### Embodiment 5

The basic structure is identical to the embodiment 2 and embodiment 3. Tension sensors of various sizes within range of 1 nm-100 μm are adhered on an internal or external side of the tube-network. The sensitivity of the tension sensor is within range of 10⁻¹⁰-10¹⁰ Newtons. The sensor senses levels or intensity of the tension on a surface of the ventricle, and transmits signals via a wire in a hollow tube inside the tube-network to a multi-channel recorder, to achieve a real-time, dynamic and continuous monitoring of ventricular wall tension, furthermore, indirectly deduce the level and variation of the tension in cardiac chamber wall.

### Embodiment 6

The basic structure is identical to the embodiment 2 and embodiment 3. PH sensors of various sizes within range of 1 nm-100 μm are adhered on an internal or external side of the tube-network. Sensitivity of the PH sensor is between 10¹⁰-10¹⁰. The sensor senses variation of PH on a internal or external surface of the cardiac chamber, and transmits signals via wire in a hollow tube inside the tube-network to a multi-channel recorder, to achieve a real-time, dynamic and continuous monitoring of PH of ventricular internal or external surface, furthermore, indirectly deduce the level and variation of the metabolic condition of myocardium in the cardiac chamber wall.

### Embodiment 7

The basic structure is identical to the Embodiment 2 and Embodiment 3. Color sensors with various sizes within range of 1 nm-100 μm are adhered on an internal side or an external side of the tube-network. Sensitivity of the color sensor is within range of 10¹⁰-10¹⁰ m optical wave. The color sensor senses color variation on the surface of ventricle, and transmits signals via a wire in a hollow tube inside the tube-network to a multi-channel recorder, to achieve a real-time, dynamic and continuous monitoring of color on the internal or external surface of ventricle, furthermore, indirectly deduce the level and variation of the severity of ventricle ischemic condition. In general, the more severe is the myocardial ischemia, lighter is the color of cardiac muscle in this part; the more is the perfusion of the oxygenated blood in cardiac muscle, the more red is the color of cardiac muscle in this part; and the more is the perfusion of the deoxygenated blood in cardiac muscle, the more purple and dark is the color of cardiac muscle in this part.

### Embodiment 8

The basic structure is identical to the Embodiment 2 and Embodiment 3. Flow sensors of various sizes within range of 1 nm-100 μm are adhered on an internal or an external side of the tube-network. The sensitivity of the flow sensor ranges within 10¹⁰-10¹⁰ L/min. The function of flow sensor is to sense blood flow of the cardiac chambers, and transmits signals via wire in a hollow tube inside the tube-network to a multi-channel recorder, to achieve a real-time, dynamic and continuous monitoring the blood flow of the cardiac chambers, furthermore, indirectly deduce the severity of ventricle ischemic condition. In general, the severity of myocardial ischemia is inversely related to both the blood flow in ventricles and cardiac function, i.e., in the case of severe myocardial ischemia, the more severe is the myocardial ischemia, the lower would be the flow rate in the cardiac chamber, and resultantly the poorer would be cardiac function.

### Embodiment 9

The basic structure is identical to the Embodiment 2 and Embodiment 3. Temperature sensors with various sizes within range of 1 nm-100 μm are adhered on an internal or external side of the tube-network. Sensitivity of the color sensor ranges within 10¹⁰-10¹⁰° C. The temperature sensor senses temperature variation on the internal or external surface of ventricle, and transmits signals via a wire in a hollow tube inside the tube-network to a multi-channel recorder, to achieve a real-time, dynamic and continuous monitoring of temperature on the internal or external surface of ventricle, furthermore, indirectly deduce the severity of ventricle ischemic condition. In general, the higher is the degree of myocardial ischemia, the lower would be temperature in this specific part of cardiac muscle; meanwhile, the more is the perfusion of oxygenated blood in cardiac muscle, the higher would be the temperature of cardiac muscle in this part.

### Embodiment 10

The basic structure is identical to the Embodiment 2 and Embodiment 3. Cardiac-electric conduction electrode of various sizes within range of 1 nm-100 μm is adhered on an internal or external side of the tube-network. A sensitivity of the cardiac-electric conduction electrode-ranges from 10¹⁰ to 10¹⁰ V. The function of cardiac-electric conduction electrode is to sense levels or variations of the voltage on an internal or external surface of the cardiac chamber, and transmits signals via a wire in a hollow tube inside the tube-network to a multi-channel recorder to achieve a real-time, dynamic and continuous monitoring of the voltage of the internal or external surface of the cardiac chamber.

### Embodiment 11

Basic structure is identical to the Embodiment 2 and Embodiment 3. Magnetic field sensor of various sizes within the range of 1 nm-100 μm is adhered on an internal side or an external side of the tube-network. Sensitivity of the magnetic field sensor is within range of 10¹⁰-10¹⁰ Tesla. The function of magnetic field sensor is to sense the magnetic field on a internal or external surface of the cardiac chamber, and transmits signals via a wire in a hollow tube inside the tube-network to a multi-channel recorder, to achieve a real-time, dynamic and continuous monitoring of the magnetic field of the internal or external surface of the cardiac chamber.

### Embodiment 12

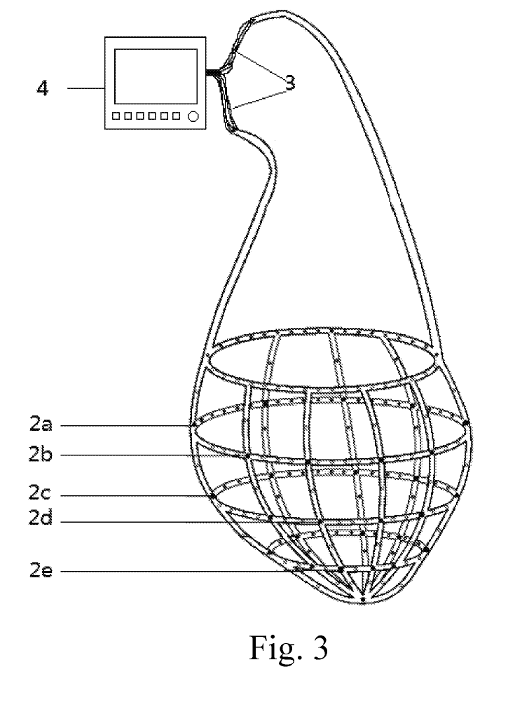

At least two types of structures in the Embodiments 4-11, comprise at least two types of sensors which may be a pressure sensor, a PH sensor, color sensor, temperature sensor, flow sensor or a cardiac-electric conduction electrode. Wires from multiple sensors pass through hollow tubes in a tube-network to transmit signals to a multi-channel electrophysiology recorder. The structure is as shown in FIG. 3. 1—cardiac tube-network; 2a—tension sensor, pressure sensor or flow sensor; 2b—cardiac-electric conduction electrode; 2c—color sensor; 2d—PH sensor; 2e—temperature sensor; 3—wire; 4—multi-channel physiology recorder (cardiac function monitor device).

### Embodiment 13

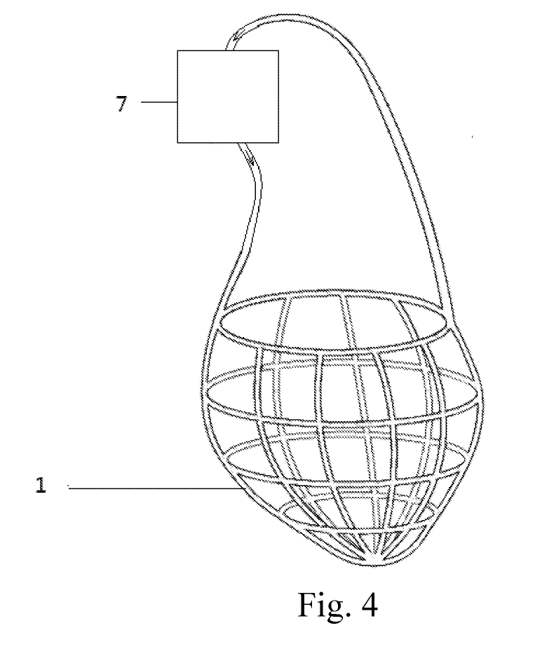

Surface attached cardiac function intervention system comprises of cardiac tube-network and a liquid perfusion device. The cardiac tube-network is adhered on the internal or external surface of one or more cardiac chambers. The tube-network is composed of hollow tubes. All the hollow tubes are completely communicated or form a plurality of independent regions, which is intercommunicated within the region, but it is not communicated between the regions. The hollow tube serves as a liquid transmission tube for the pressure intervention, and end of the tube-network is connected with an external liquid perfusion device. The structure is as shown in FIG. 4: 1—cardiac tube-network; 7—liquid perfusion device.

### Embodiment 14

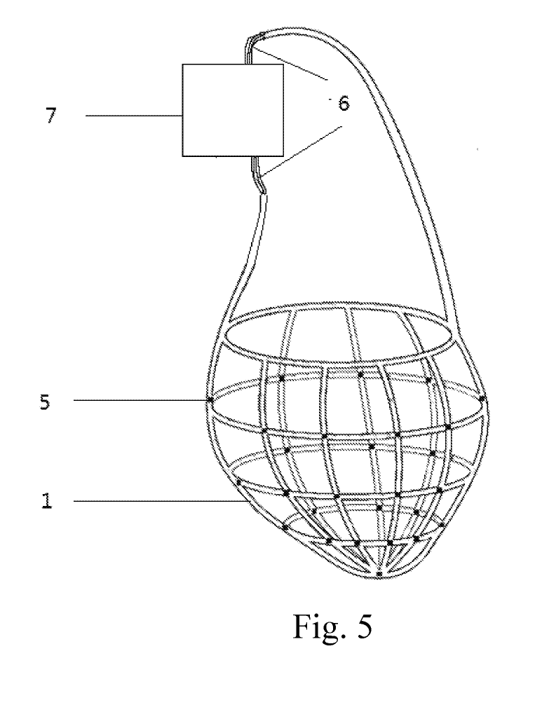

Surface attached cardiac function intervention system comprises of a cardiac tube-network, electrical/magnetic stimulation devices, an electrical/magnetic power output device and wire. The cardiac tube-network is adhered on an internal or external surface of one or more cardiac chambers. The tube-network is composed of hollow tubes. All of the hollow tubes are completely communicated or form a plurality of independent regions, which is intercommunicated within the region, but it is not communicated between the regions. An electrical/magnetic stimulation device is adhered on an internal or external surface of the tube-network, passes through a wire in a hollow tube inside the tube-network to connect an electrical/magnetic power output device. The structure is as shown in FIG. 5, wherein 1—cardiac tube-network; 5—electrical/magnetic stimulation device; 6—wire; 7—electrical/magnetic power output device.

### Embodiment 15

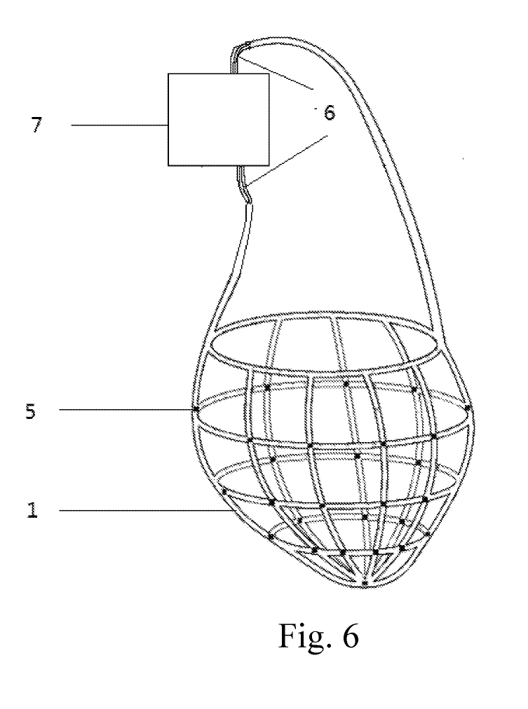

Surface attached cardiac function intervention system comprises of a cardiac tube-network, medicine loading devices, microsyringes and medicine delivery tubes. Cardiac tube-network is adhered on an internal or external surface of one or more cardiac chambers. The tube-network is composed of hollow tubes. All of the hollow tubes are completely communicated or form a plurality of independent regions, which is intercommunicated within the region, but it is not communicated between the regions. The medicine intervention device comprises of a medicine loading device and a microsyringe connected with the medicine loading device by a medicine delivery tube. The microsyringe is adhered on an internal or external surface of the tube-network. Tube-network is hollow and acts as a delivery tube or the medicine delivery tube of the microsyringe passes through the hollow tube inside the tube-network to connect with an external medicine loading device. The structure is as shown in FIG. 6, wherein 1—cardiac tube-network, 5—microsyringe; 6—medicine delivery tube; 7—medicine loading device.

### Embodiment 16

Surface attached cardiac function intervention system comprises of at least two structures selected from the embodiments 13-15 such as a pressure intervention device, an electrical/magnetic stimulation device and a medicine intervention device. When the hollow tubes of tube-network serves as a liquid delivery tube of the pressure intervention device or a medicine delivery tube of the medicine intervention device, or wires of electrical/magnetic intervention or stimulation device, other wires or delivery tubes could be distributed on an internal or external side of the tube-network, so as to be connected with an external device via tube-network end.

### Embodiment 17

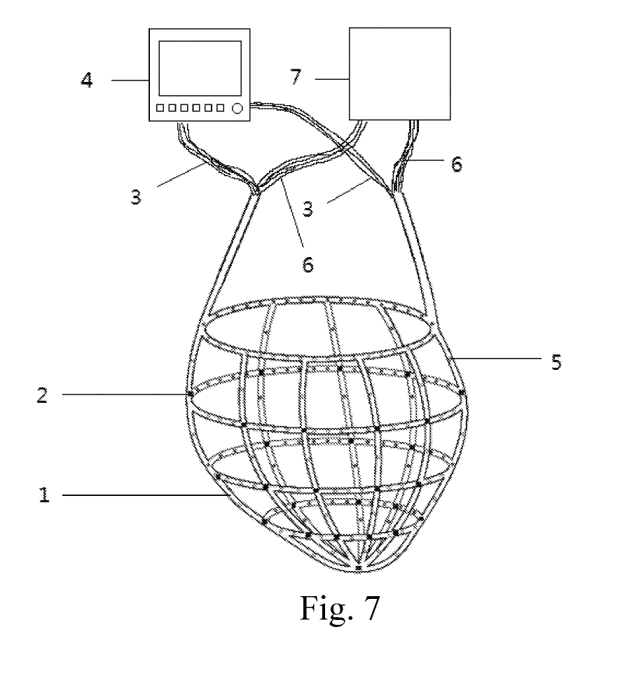

A cardiac function monitor and intervention system attached outside or inside of the heart comprises of at least one structure selected from the embodiments 3-12 plus at least one structure selected from embodiments 13-16, whose structure is as shown in FIG. 7, wherein 1—cardiac tube-network; 2—physiological and biochemical sensor; 3—wire of the physiological and biochemical sensor, 4—cardiac function monitor device; 5—microsyringe or electrical/magnetic stimulation electrode; 6—medicine delivery tube and/or wire of the electrical/magnetic stimulation device and/or liquid delivery tube; 7—medicine loading device and/or power output device and/or liquid perfusion device.

### Embodiment 18

Surface attached cardiac function intervention system from the embodiments 1-17 comprises of two or more components. One component could be set inside or outside another one or ones to get better or more inward force. Outside component or components is or are harder than inside one or ones.

What we claimed is:

1. A surface attached cardiac function monitor and/or intervention system comprising a cardiac support device and a cardiac function monitor device and/or a intervention device;
wherein the cardiac support device adheres on an internal or external surface of a cardiac chamber and supports the cardiac chambers;
the cardiac function monitor device is connected with a physiological and biochemical sensor;
the intervention device has at least one component selected from a pressure intervention device, an electrical/magnetic stimulation intervention device or a medicine intervention device; wherein the pressure intervention device comprises a liquid delivery tube and a liquid perfusion device; the electrical/magnetic stimulation intervention device comprises an intervention or stimulation electrical/magnetic device, wires and a power output device; the medicine intervention device comprises a microsyringe, a medicine loading device, and medicine delivery tubes;
one component selected from the physiological and biochemical sensors, the liquid delivery tubes, the intervention or stimulation electrical/magnetic devices or the microsyringes is embedded in the tube walls, filled in the aperture of the tube walls or adhered on an internal or external surface of the cardiac support device.

2. The system, as recited in claim 1, wherein the cardiac support device is a cardiac tube-network.

3. The system, as recited in claim 2, wherein the cardiac tube-network is a soft, elastic and end-sealed tube-network made of hollow tubes, which are completely communicated or form a plurality of independent areas, and the interior of each independent area is intercommunicating while the independent areas are not communicating with each other, the cardiac tube-network has at least one open end extending out of human body.

4. The system, as recited in claim 1, wherein the physiological and biochemical sensor transmits variations in physiological and biochemical parameters to the cardiac function monitor device in vitro; wherein the physiological and biochemical parameters on internal or external surface of the heart are cardiac-electric voltage, PH value, temperature, color, cardiac wall tension, cardiac chamber internal pressure, blood flow of cardiac chamber and hemodynamics of cardiac chamber.

5. The system, as recited in claim 1, wherein the cardiac function monitor device is selected from a cardiac-electric monitor device and a multi-channel physiological recorder.

6. The system, as recited in claim 1, wherein the physiological and biochemical sensor is a tension sensor, a pressure sensor, a PH sensor, a color sensor, a temperature sensor, a flow sensor and a cardiac-electric conduction electrode.

7. The system, as recited in claim 1, wherein an output of the electrical/magnetic stimulation intervention device is electric energy or electromagnetic energy.

8. The system, as recited in claim 1, wherein the medicine is selected from any group of medicine like diuretic, cardiotonic, angiotensin-converting enzyme inhibitor, angiotensin II receptor blocker, β-adrenergic blocker, anticoagulant, vasodilator, anti-myocardial ischemia drug, coronary-dilating drug and stem cells.

9. The system, as recited in claim 8, wherein the medicine is selected from the group of: sodium ferulate injection, esmolol hydrochloride injection, composite salvia miltiorrhiza injection, ligustrazine injection, breviscapine injection, safflower injection, shuxuening injection, buflomedil hydrochloride injection, puerarin injection, ginkgo dipyridamole injection, ligustrazine glucose injection, astragalus injection, Shenmai injection, nitroglycerin injection, isosorbide dinitrate injection, low molecular weight heparin calcium injection, fibrinolysis enzyme for injection, defibrase for injection, urokinase for injection, cardiac stem cells, bone marrow stem cells and embryonic stem cells.

10. The system, as recited in claim 1, wherein the cardiac support device is made of conductive material of hydrogel, silica gel or degradable biocompatible materials.
